(19) United States
(12) Patent Application Publication
(10) Pub. No.: US 2011/0023147 A1
(43) Pub. Date: Jan. 27, 2011

(54) GENOMIC EDITING OF PRION DISORDER-RELATED GENES IN ANIMALS

(75) Inventors: Edward Weinstein, St. Louis, MO (US); Phil Simmons, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,713

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/48; A01K 67/027; C12N 5/10

(52) U.S. Cl. 800/3; 800/14; 800/15; 800/16; 800/9; 435/325; 435/350; 435/351; 435/352; 435/363; 435/353; 800/17

(57) ABSTRACT

The present invention provides genetically modified animals and cells comprising edited chromosomal sequences encoding proteins that are associated with cognitive disorders. In particular, the animals or cells are generated using a zinc finger nuclease-mediated editing process. Also provided are methods of using the genetically modified animals or cells disclosed herein to screen agents for toxicity and other effects.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding a prion disorder-related protein. In particular, the invention relates to the use of a zinc finger nuclease-mediated process to edit chromosomal sequences encoding prion disorder-related proteins in animals or cells.

### BACKGROUND OF THE INVENTION

Prion disorders, also known as Transmissible spongiform encephalopathies (TSEs), are a group of rare fatal neurodegenerative diseases affecting humans and other animals. The hallmark event in all TSEs appears to be the misfolding of the prion protein (PrP). The conversion of PrP^{C}, the normal PrP, to the pathological isoform, PrP^{Sc} involves a conformational change with no gene sequence or post-translation differentiation. With a concomitant loss of enzyme function, PrP misfolding leads to the accumulation of protease-resistant insoluble deposits in the brain.

Human PrP is the product of a single gene, Prnp, which directs the synthesis of a protein containing 253 amino acid residues. In addition, a glycosylphosphatidylinositol anchor (GPI) attaches the protein to the outer surface of the cell membrane. All of the known prion disorders in animals and humans lead to the accumulation of abnormal prion protein aggregates in the central nervous system, sometimes in the form of amyloid plaques similar to the lesions found in other neurodegenerative illnesses, such as Alzheimer's disease. The coding sequences of mammalian prion protein genes are highly conserved. In humans, a polymorphism at PRNP codon 129, between methionine and valine, has a strong disease susceptibility and phenotype modifying effect. However, the progress of ongoing research into the causes and treatments of these prion disorders is hampered by the onerous task of developing an animal model which incorporates the genes proposed to be involved in the development or severity of the disorders.

Conventional methods such as gene knockout technology may be used to edit a particular gene in a potential model organism in order to develop an animal model of a prion disorder. However, gene knockout technology may require months or years to construct and validate the proper knockout models. In addition, genetic editing via gene knockout technology has been reliably developed in only a limited number of organisms such as mice. Even in a best case scenario, mice typically show low intelligence, which related to cognition, psychiatric disease, and behavior; and their genetic background influence the incubation time with prion. Incubation time is a quantitative trait used for genetic and phenotypic characterization, and gene mapping. Not limited by the above reasons, it generally makes mice a poor choice of organism in which to study complex disorders of neurotransmission, such as prion disorders. Ideally, the selection of organism in which to model a complex neurotransmission disorder should be based on the organism's ability to exhibit the characteristics of the disorder as well as its amenability to existing research methods.

The rat is emerging as a genetically malleable, preferred model organism for the study of neurotransmission disorders, particularly because these disorders are not well-modeled in mice. Rats are a superior choice compared to mice as model organisms for the study of human diseases of neurotransmission, psychiatric disease, and behavior due to their higher intelligence, complex behavioral repertoire, and observable responses to behavior-modulating drugs, all of which better approximate the human and other animal condition. Further, the larger physical size of rats relative to mice facilitates experimentation that requires dissection, in vivo imaging, or isolation of specific cells or organ structures for cellular or molecular studies of these prion disorders.

A need exists for animals with modification to one or more genes associated with prion disorders to be used as model organisms in which to study these disorders. The genetic modifications may include gene knockouts, expression, modified expression, or over-expression of alleles that either cause or are associated with prion disorders in humans. Further, a need exists for modification of one or more genes associated with prion disorder in a variety of organisms in order to develop appropriate animal models of neurotransmission diseases such as Inherited Human Transmissible Spongiform Encephalopathies, Prion-associated disorders, Prion-Induced Disorders, Prion Protein Diseases, Transmissible Dementias, Creutzfeldt-Jakob disease (CJD), fatal insomnia, Gerstmann-Straussler syndrome (GSS) and kuru in humans, as well as bovine spongiform encephalopathy (BSE), ovine scrapie, and chronic wasting disease in animals.

### SUMMARY OF THE INVENTION

One aspect of the present disclosure encompasses a genetically modified animal comprising at least one edited chromosomal sequence encoding a prion disorder-related protein.

A further aspect provides a non-human embryo comprising at least one RNA molecule encoding a zinc finger nuclease that recognizes a chromosomal sequence encoding a prion disorder-related protein, and, optionally, at least one donor polynucleotide comprising a sequence encoding an ortholog of the prion disorder-related protein.

Another aspect provides an isolated cell comprising at least one edited chromosomal sequence encoding a prion disorder-related protein.

Yet another aspect encompasses a method for assessing the effect of an agent in an animal. The method comprises contacting a genetically modified animal comprising at least one edited chromosomal sequence encoding a prion disorder-related protein with the agent, and comparing results of a selected parameter to results obtained from contacting a wild-type animal with the same agent. The selected parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) efficacy of the agent or its metabolite(s).

Still yet another aspect encompasses a method for assessing the therapeutic potential of an agent in an animal. The method includes contacting a genetically modified animal comprising at least one edited chromosomal sequence encoding a prion disorder-related protein with the agent, and comparing the results of a selected parameter to results obtained from a wild-type animal with no contact with the same agent. The selected parameter may be chose from a) spontaneous behaviors; b) performance during behavioral testing; c) physiological anomalies; d) abnormalities in tissues or cells; e) biochemical function; f) molecular structures; g) differential responses to an agent.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with prion disorder. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with prion disorder generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with prion disorder using targeted zinc finger nuclease technology is rapid, precise, and highly efficient.

### (I) Genetically Modified Animals

One aspect of the present disclosure provides a genetically modified animal in which at least one chromosomal sequence encoding a prion disorder-related protein has been edited. For example, the edited chromosomal sequence may be inactivated such that the sequence is not transcribed and/or a functional prion disorder-related protein is not produced. Alternatively, the edited chromosomal sequence may be modified such that it codes for an altered prion disorder-related protein. For example, the chromosomal sequence may be modified such that at least one nucleotide is changed and the expressed prion disorder-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed prion disorder-related protein comprises a single amino acid deletion or insertion, provided such a protein is functional. The modified protein may have altered substrate specificity, altered enzyme activity, altered kinetic rates, and so forth. Furthermore, the edited chromosomal sequence may comprise an integrated sequence and/or a sequence encoding an orthologous protein associated with prion disorders. The genetically modified animal disclosed herein may be heterozygous for the edited chromosomal sequence encoding a protein associated with prion disorders. Alternatively, the genetically modified animal may be homozygous for the edited chromosomal sequence encoding a protein associated with prion disorders.

In one embodiment, the genetically modified animal may comprise at least one inactivated chromosomal sequence encoding a prion disorder-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional prion disorder-related protein is not produced. The inactivated chromosomal sequence comprises no exogenously introduced sequence. Such an animal may be termed a “knockout.” Also included herein are genetically modified animals in which two, three, four, five, six, seven, eight, nine, or ten or more chromosomal sequences encoding proteins associated with prion disorders.

In another embodiment, the genetically modified animal may comprise at least one edited chromosomal sequence encoding an orthologous protein associated with prion disorders. The edited chromosomal sequence encoding an orthologous prion disorder-related protein may be modified such that it codes for an altered protein. For example, the edited chromosomal sequence encoding a prion disorder-related protein may comprise at least one modification such that an altered version of the protein is produced. In some embodiments, the edited chromosomal sequence comprises at least one modification such that the altered version of the prion disorder-related protein results in prion disorders in the animal. In other embodiments, the edited chromosomal sequence encoding a prion disorder-related protein comprises at least one modification such that the altered version of the protein protects against a prion disorder in the animal. The modification may be a missense mutation in which substitution of one nucleotide for another nucleotide changes the identity of the coded amino acid.

In yet another embodiment, the genetically modified animal may comprise at least one chromosomally integrated sequence. The chromosomally integrated sequence may encode an orthologous prion disorder-related protein, an endogenous prion disorder-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a prion disorder-related protein may be integrated into a “safe harbor” locus, such as the Rosa26 locus, HPRT locus, or AAV locus. An animal comprising a chromosomally integrated sequence encoding a prion disorder-related protein may be called a “knock-in.” The present disclosure also encompasses genetically modified animals in which two, three, four, five, six, seven, eight, nine, or ten or more sequences encoding protein(s) associated with prion disorder-related disorders are integrated into the genome.

The chromosomally integrated sequence encoding a prion disorder-related protein may encode the wild type form of the protein. Alternatively, the chromosomally integrated sequence encoding a prion disorder-related protein may comprise at least one modification such that an altered version of the protein is produced. In some embodiments, the chromosomally integrated sequence encoding a prion disorder-related protein comprises at least one modification such that the altered version of the protein produced causes a prion disorder. In other embodiments, the chromosomally integrated sequence encoding a prion disorder-related protein comprises at least one modification such that the altered version of the protein protects against the development of a prion disorder.

In an additional embodiment, the genetically modified animal may be a “humanized” animal comprising at least one chromosomally integrated sequence encoding a functional human prion disorder-related protein. The functional human prion disorder-related protein may have no corresponding ortholog in the genetically modified animal. Alternatively, the wild-type animal from which the genetically modified animal is derived may comprise an ortholog corresponding to the functional human prion disorder-related protein. In this case, the orthologous sequence in the “humanized” animal is inactivated such that no functional protein is made and the “humanized” animal comprises at least one chromosomally integrated sequence encoding the human prion disorder-related protein. Those of skill in the art appreciate that “humanized” animals may be generated by crossing a knock out animal with a knock in animal comprising the chromosomally integrated sequence.

In yet another embodiment, the genetically modified animal may comprise at least one edited chromosomal sequence encoding a prion disorder-related protein such that the expression pattern of the protein is altered. For example, regulatory regions controlling the expression of the protein, such as a promoter or transcription binding site, may be altered such that the prion disorder-related protein is over-produced, or the tissue-specific or temporal expression of the protein is altered, or a combination thereof. Alternatively, the expression pattern of the prion disorder-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a prion disorder-related protein. The genetically modified animal comprising the lox-flanked chromosomal sequence encoding a prion disorder-related protein may then be crossed with another genetically modified animal expressing Cre recombinase. Progeny animals comprising the lox-flanked chromosomal sequence and the Cre recombinase are then produced, and the lox-flanked chromosomal sequence encoding a prion disorder-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a prion disorder-related protein.

### (a) Prion Disorder-Related Proteins

The prion disorders are now among the best understood of the degenerative brain diseases. Prion disorders appear to be diseases of protein conformation, which results in abnormal protein aggregation. It is believed that the mechanism of prion replication and the potential biological function of normal PrP^{C}, and other signaling proteins that bind to PrP^{C} may be involved in the prion disorders. Stress inducible protein 1 (STI1) is a potential PrP^{C}-interacting protein in the plasma membrane. Other candidate ligands for PrP^{C} include Bcl-2, Hsp60, Bax-inhibiting peptide, nuclear respiratory factor 2, apolipoprotein A1, neural cell-adhesion molecules, heparin, laminin and laminin receptor. PrP fragment 106-126 is believed to suppress a neurotropic signal by competing for the interaction between PrP^{C} and its endogenous ligand, which otherwise constitutively transduces a survival signal through the PKA pathway. In addition, the conformational changes associated with PrP conversion result in decreased affinity for the ligand, thus eliminating the PrPC-mediated neuroprotective effect.

Prnd, a gene located downstream of PrP, which encodes the 179-residues protein Doppel (Dpl). Some of the TSE clinical symptoms, such as ataxia, might be due to increased or unscheduled expression and activity of Dpl in brain during the disease. Only in the absence of PrP^{C} is Dpl able to bind the ligand, resulting in neurotoxicity. Therefore, it is believed that Dpl plays a role in peripheral prion replication and neuroinvasion, especially considering that Prnd is expressed in spleen, an organ implicated in peripheral prion pathogenesis.

It is also noted that phenotypic diversity in human prion disease relates in part to the propagation of disease-related PrP isoforms with distinct physicochemical properties. Mutated PrPs appears to be able to fold into a number of different pathogenic conformers, which differ in toxicity or neuropathological targeting. PRNP mutations include point mutations leading to amino-acid substitution or premature stop codon, and insertion. A non-limiting example is the polymorphic codon 129 genotype of the PrP gene. Various mutation correlates to clinical observation ranging from age of onset, duration of disease to clinical phenotype.

Another function of PrP^{C} is in the binding and metabolism of copper. PrP misfolding might result in abnormal brain metal levels, leading to neuronal damage. In addition, a GPI-anchored PrP^{C} can initiate a signaling cascade although the molecular bases remain unclear.

Therefore there are a diverse set of proteins associated with susceptibility for developing a prion disorder, the presence of a prion disorder, the severity of a prion disorder or any combination thereof. Another non-limiting example is HECTD2 gene, which encodes an E3-ubiquitin ligase protein and has been linked to prion disease incubation time in mice and a susceptibility haplotype has been associated with human prion disease. The ubiquitin-proteosome system is a common molecular mechanisms and pathways implicated in the pathogenesis of several neurodegenerative diseases, such as AD, prion diseases and other neurodegenerative disorders. Prion diseases have been shown to share some of the neuropathologic hallmarks of AD, in that, the abnormal forms of PrP accumulate in the brain often in the form of amyloid plaques.

Non-limiting examples of a prion disorder include Inherited Human Transmissible Spongiform Encephalopathies, Prion-associated disorders, Prion-Induced Disorders, Prion Protein Diseases, Transmissible Dementias, Creutzfeldt-Jakob disease (CJD), fatal insomnia, Gerstmann-Straussler syndrome (GSS) and kuru in humans, as well as bovine spongiform encephalopathy (BSE), ovine scrapie, and chronic wasting disease in animals. The prion disorders also related to certain type of symptoms include but not limited to personality changes, psychiatric problems, depression, lack of coordination, unsteady gait, myoclonus, unusual sensations, insomnia, confusion, memory problems, severe mental impairment, inability to move, inability to speak.

The prion disorder-related proteins or isoforms thereof are typically selected based on an experimental association of the prion disorder-related protein or isoform to a prion disorder. For example, the production rate or circulating concentration of a prion disorder-related protein or isoform may be elevated or depressed in a population having a prion disorder relative to a population lacking the prion disorder. Differences in protein or certain isoform levels may be assessed using proteomic techniques including but not limited to Western blot, immunohistochemical staining, enzyme linked immunosorbent assay (ELISA), and mass spectrometry. Alternatively, the prion disorder-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

Non-limiting examples of prion disorder-related proteins include PrP^{C} and its isoforms, PrP^{Sc} and its isoforms, HECTD2 (e3-ubipuitin ligase protein), STI1 (stress inducible protein 1), DPL (residue Doppel protein, encoded by Prnd), APOA1 (Apolipoprotein A1), BCL-2 (B-cell lymphoma 2), HSP60 (Heat shock 60 kDa protein), BAX-inhibiting peptide (Bcl-2-associated X protein inhibitor), NRF2 (nuclear respiratory factor 2), NCAMs (neural cell-adhesion molecules), heparin, laminin and laminin receptor.

Further, non-limiting example of genes that may related to neurodegenerative conditions in prion disorders include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), ADRA1D (Alpha-1D adrenergic receptor for Alpha-1D adrenoreceptor), AHSG (Alpha-2-HS-glycoprotein), AlF1 (Allograft inflammatory factor 1), ALAS2 (Delta-aminolevulinate synthase 2), AMBP (Alpha-1-microglobulin/bikunin precursor), ANK3 (Ankryn 3), ANXA3 (Annexin A3), APCS (Amyloid P component serum), APOA1 (Apolipoprotein A1), APOA12 (Apolipoprotein A2), APOB (Apolipoprotein B), APOC1 (Apolipoprotein C1), APOE (Apolipoprotein E), APOH (Apolipoprotein H), APP (Amyloid precursor protein), ARC (Activity-regulated cytoskeleton-associated protein), ARF6 (ADP-ribosylation factor 6), ARHGAP5 (Rho GTPase activating protein 5), ASCL1 (Achaete-scute homolog 1), B2M (Beta-2 microglobulin), B4GALNT1 (Beta-1,4-N-acetyl-galactosaminyl transferase 1), BAX (Bcl-2-associated X protein), BCAT (Branched chain amino-acid transaminase 1 cytosolic), BCKDHA (Branched chain keto acid dehydrogenase E1 alpha), BCKDK (Branched chain alpha-ketoacid dehydrogenase kinase), BCL2 (B-cell lymphoma 2), BCL2L1 (BCL2-like 1), BDNF (Brain-derived neurotrophic factor), BHLHE40 (Class E basic helix-loop-helix protein 40), BHLHE41 (Class E basic helix-loop-helix protein 41), BMP2 (Bone morphogenetic protein 2A), BMP3 (Bone morphogenetic protein 3), BMP5 (Bone morphogenetic protein 5), BRD1 (Bromodomain containing 1), BTC (Betacellulin), BTNL8 (Butyrophilin-like protein 8), CALB1 (Calbindin 1), CALM1 (Calmodulin 1), CAMK1 (Calcium/calmodulin-dependent protein kinase type I), CAMK4 (Calcium/calmodulin-dependent protein kinase type IV), CAMKIIB (Calcium/calmodulin-dependent protein kinase type IIB), CAMKIIG (Calcium/calmodulin-dependent protein kinase type IIG), CASP11 (Caspase-10), CASP8 (Caspase 8 apoptosis-related cysteine peptidase), CBLN1 (cerebellin 1 precursor), CCL2 (Chemokine (C-C motif) ligand 2), CCL22 (Chemokine (C-C motif) ligand 22), CCL3 (Chemokine (C-C motif) ligand 3), CCL8 (Chemokine (C-C motif) ligand 8), CCNG1 (Cyclin-G1), CCNT2 (Cyclin T2), CCR4 (C-C chemokine receptor type 4 (CD194)), CD58 (CD58), CD59 (Protectin), CD5L (CD5 antigen-like), CD93 (CD93), CDKN2AIP (CDKN2A interacting protein), CDKN2B (Cyclin-dependent kinase inhibitor 2B), CDX1 (Homeobox protein CDX-1), CEA (Carcinoembryonic antigen), CEBPA (CCAAT/enhancer-binding protein alpha), CEBPB (CCAAT/enhancer binding protein C/EBP beta), CEBPB (CCAAT/enhancer-binding protein beta), CEBPD (CCAAT/enhancer-binding protein delta), CEBPG (CCAAT/enhancer-binding protein gamma), CENPB (Centromere protein B), CGA (Glycoprotein hormone alpha chain), CGGBP1 (CGG triplet repeat-binding protein 1), CHGA (Chromogranin A), CHGB (Secretoneurin), CHN2 (Beta-chimaerin), CHRD (Chordin), CHRM1 (Cholinergic receptor muscarinic 1), CITED2 (Cbp/p300-interacting transactivator 2), CLEC4E (C-type lectin domain family 4 member E), CMTM2 (CKLF-like MARVEL transmembrane domain-containing protein 2), CNTN1 (Contactin 1), CNTNAP1 (Contactin-associated protein-like 1), CR1 (Erythrocyte complement receptor 1), CREM (cAMP-responsive element modulator), CRH (Corticotropin-releasing hormone), CRHR1 (Corticotropin releasing hormone receptor 1), CRKRS (Cell division cycle 2-related protein kinase 7), CSDA (DNA-binding protein A), CSF3 (Granulocyte colony stimulating factor 3), CSF3R (Granulocyte colony-stimulating factor 3 receptor), CSP (Chemosensory protein), CSPG4 (Chondroitin sulfate proteoglycan 4), CTCF (CCCTC-binding factor zinc finger protein), CTGF (Connective tissue growth factor), CXCL12 (Chemokine C-X-C motif ligand 12), DAD1 (Defender against cell death 1), DAXX (Death associated protein 6), DBN1 (Drebrin 1), DBP (D site of albumin promoter-albumin D-box binding protein), DDR1 (Discoidin domain receptor family member 1), DDX14 (DEAD/DEAN box helicase), DEFA3 (Defensin alpha 3 neutrophil-specific), DVL3 (Dishevelled dsh homolog 3), EDN1 (Endothelin 1), EDNRA (Endothelin receptor type A), EGF (Epidermal growth factor), EGFR (Epidermal growth factor receptor), EGR1 (Early growth response protein 1), EGR2 (Early growth response protein 2), EGR3 (Early growth response protein 3), EIF2AK2 (Eukaryotic translation initiation factor 2-alpha kinase 2), ELANE (Elastase neutrophil expressed), ELK1 (ELK1 member of ETS oncogene family), ELK3 (ELK3 ETS-domain protein (SRF accessory protein 2)), EML2 (Echinoderm microtubule associated protein like 2), EPHA4 (EPH receptor A4), ERBB2 (V-erb-b2 erythroblastic leukemia viral oncogene homolog 2), ERBB3 (Receptor tyrosine-protein kinase erbB-3), ESR2 (Estrogen receptor 2), ESR2 (Estrogen receptor 2), ETS1 (V-ets erythroblastosis virus E26 oncogene homolog 1), ETV6 (Ets variant 6), FASLG (Fas ligand TNF superfamily member 6), FCAR (Fc fragment of IgA receptor), FCER1G (Fc fragment of IgE high affinity I receptor for gamma polypeptide), FCGR2A (Fc fragment of IgG low affinity IIa receptor—CD32), FCGR3B (Fc fragment of IgG low affinity IIIb receptor—CD16b), FCGRT (Fc fragment of IgG receptor transporter alpha), FGA (Basic fibrinogen), FGF1 (Acidic fibroblast growth factor 1), FGF14 (Fibroblast growth factor 14), FGF16 (fibroblast growth factor 16), FGF18 (Fibroblast growth factor 18), FGF2 (Basic fibroblast growth factor 2), FIBP (Acidic fibroblast growth factor intracellular binding protein), FIGF (C-fos induced growth factor), FMR1 (Fragile X mental retardation 1), FOSB (FBJ murine osteosarcoma viral oncogene homolog B), FOXO1 (Forkhead box O1), FSHB (Follicle stimulating hormone beta polypeptide), FTH1 (Ferritin heavy polypeptide 1), FTL (Ferritin light polypeptide), G1P3 (Interferon alpha-inducible protein 6), G6S (N-acetylglucosamine-6-sulfatase), GABRA2 (Gamma-aminobutyric acid A receptor alpha 2), GABRA3 (Gamma-aminobutyric acid A receptor alpha 3), GABRA4 (Gamma-aminobutyric acid A receptor alpha 4), GABRB1 (Gamma-aminobutyric acid A receptor beta 1), GABRG1 (Gamma-aminobutyric acid A receptor gamma 1), GADD45A (Growth arrest and DNA-damage-inducible alpha), GCLC (Glutamate-cysteine ligase catalytic subunit), GDF15 (Growth differentiation factor 15), GDF9 (Growth differentiation factor 9), GFRA1 (GDNF family receptor alpha 1), GIT1 (G protein-coupled receptor kinase interactor 1), GNA13 (Guanine nucleotide-binding protein/G protein alpha 13), GNAQ (Guanine nucleotide binding protein/G protein q polypeptide), GPR12 (G protein-coupled receptor 12), GPR18 (G protein-coupled receptor 18), GPR22 (G protein-coupled receptor 22), GPR26 (G protein-coupled receptor 26), GPR27 (G protein-coupled receptor 27), GPR77 (G protein-coupled receptor 77), GPR85 (G protein-coupled receptor 85), GRB2 (Growth factor receptor-bound protein 2), GRLF1 (Glucocorticoid receptor DNA binding factor 1), GST (Glutathione S-transferase), GTF2B (General transcription factor IIB), GZMB (Granzyme B), HAND1 (Heart and neural crest derivatives expressed 1), HAVCR1 (Hepatitis A virus cellular receptor 1), HES1 (Hairy and enhancer of split 1), HES5 (Hairy and enhancer of split 5), HLA-DQA1 (Major histocompatibility complex class II DQ alpha), HOXA2 (Homeobox A2), HOXA4 (Homeobox A4), HP (Haptoglobin), HPGDS (Prostaglandin-D synthase), HSPA8 (Heat shock 70 kDa protein 8), HTR1A (5-hydroxytryptamine receptor 1A), HTR2A (5-hydroxytryptamine receptor 2A), HTR3A (5-hydroxytryptamine receptor 3A), ICAM1 (Intercellular adhesion molecule 1 (CD54)), IFIT2 (Interferon-induced protein with tetratricopeptide repeats 2), IFNAR2 (Interferon alpha/beta/omega receptor 2), IGF1 (Insulin-like growth factor 1), IGF2 (Insulin-like growth factor 2), IGFBP2 (Insulin-like growth factor binding protein 2, 36 kDa), IGFBP7 (Insulin-like growth factor binding protein 7), IL10 (Interleukin 10), IL10RA (Interleukin 10 receptor alpha), IL11 (Interleukin 11), IL11RA (Interleukin 11 receptor alpha), IL11RB (Interleukin 11 receptor beta), IL13 (Interleukin 13), IL15 (Interleukin 15), IL17A (Interleukin 17A), IL17RB (interleukin 17 receptor B), IL18 (Interleukin 18), IL18RAP (Interleukin 18 receptor accessory protein), IL1R2 (Interleukin 1 receptor type II), IL1RN (Interleukin 1 receptor antagonist), IL2RA (Interleukin 2 receptor alpha), IL4R (Interleukin 4 receptor), IL6 (Interleukin 6), IL6R (Interleukin 6 receptor), IL7 (Interleukin 7), IL8 (Interleukin 8), IL8RA (Interleukin 8 receptor alpha), IL8RB (Interleukin 8 receptor beta), ILK (Integrin-linked kinase), INPP4A (Inositol polyphosphate-4-phosphatase type I, 107 kDa), INPP4B (Inositol polyphosphate-4-phosphatase type I beta), INS (Insulin), IRF2 (Interferon regulatory factor 2), IRF3 (Interferon regulatory factor 3), IRF9 (Interferon regulatory factor 9), IRS1 (Insulin receptor substrate 1), ITGA4 (integrin alpha 4), ITGA6 (Integrin alpha-6), ITGAE (Integrin alpha E), ITGAV (Integrin alpha-V), JAG1 (Jagged 1), JAK1 (Janus kinase 1), JDP2 (Jun dimerization protein 2), JUN (Jun oncogene), JUNB (Jun B proto-oncogene), KCNJ15 (Potassium inwardly-rectifying channel subfamily J member 15), KIF5B (Kinesin family member 5B), KLRC4 (Killer cell lectin-like receptor subfamily C member 4), KRT8 (Keratin 8), LAMP2 (Lysosomal-associated membrane protein 2), LEP (Leptin), LHB (Luteinizing hormone beta polypeptide), LRRN3 (Leucine rich repeat neuronal 3), MAL (Mal T-cell differentiation protein), MAN1A1 (Mannosidase alpha class 1A member 1), MAOB (Monoamine oxidase B), MAP3K1 (Mitogen-activated protein kinase kinase kinase 1), MAPK1 (Mitogen-activated protein kinase 1), MAPK3 (Mitogen-activated protein kinase 3), MAPRE2 (Microtubule-associated protein RP/EB family member 2), MARCKS (Myristoylated alanine-rich protein kinase C substrate), MAS1 (MAS1 oncogene), MASL1 (MAS1 oncogene-like), MBP (Myelin basic protein), MCL1 (Myeloid cell leukemia sequence 1), MDMX (MDM2-like p53-binding protein), MECP2 (Methyl CpG binding protein 2), MFGE8 (Milk fat globule-EGF factor 8 protein), MIF (Macrophage migration inhibitory factor), MMP2 (Matrix metallopeptidase 2), MOBP (Myelin-associated oligodendrocyte basic protein), MUC16 (Cancer antigen 125), MX2 (Myxovirus (influenza virus) resistance 2), MYBBP1A (MYB binding protein 1a), NBN (Nibrin), NCAM1 (Neural cell adhesion molecule 1), NCF4 (Neutrophil cytosolic factor 4 40 kDa), NCOA1 (Nuclear receptor coactivator 1), NCOA2 (Nuclear receptor coactivator 2), NEDD9 (Neural precursor cell expressed developmentally down-regulated 9), NEUR (Neuraminidase), NFATC1 (Nuclear factor of activated T-cells cytoplasmic calcineurin-dependent 1), NFE2L2 (Nuclear factor erythroid-derived 2-like 2), NFIC (Nuclear factor I/C), NFKBIA (Nuclear factor of kappa light polypeptide gene enhancer in B-cells inhibitor alpha), NGFR (Nerve growth factor receptor), NIACR2 (niacin receptor 2), NLGN3 (Neuroligin 3), NPFFR2 (neuropeptide FF receptor 2), NPY (Neuropeptide Y), NR3C2 (Nuclear receptor subfamily 3 group C member 2), NRAS (Neuroblastoma RAS viral (v-ras) oncogene homolog), NRCAM (Neuronal cell adhesion molecule), NRG1 (Neuregulin 1), NRTN (Neurturin), NRXN1 (Neurexin 1), NSMAF (Neutral sphingomyelinase activation associated factor), NTF3 (Neurotrophin 3), NTF5 (Neurotrophin 4/5), ODC1 (Ornithine decarboxylase 1), OR10A1 (Olfactory receptor 10A1), OR1A1 (Olfactory receptor family 1 subfamily A member 1), OR1N1 (Olfactory receptor family 1 subfamily N member 1), OR3A2 (Olfactory receptor family 3 subfamily A member 2), OR7A17 (Olfactory receptor family 7 subfamily A member 17), ORM1 (Orosomucoid 1), OXTR (Oxytocin receptor), P2RY13 (Purinergic receptor P2Y G-protein coupled 13), P2Y12 (Purinergic receptor P2Y G-protein coupled 12), P70S6K (P70S6 kinase), PAK1 (P21/Cdc42/Rac1-activated kinase 1), PAR1 (Prader-Willi/Angelman region-1), PBEF1 (Pre-B-cell colony enhancing factor 1), PCAF (P300/CBP-associated factor), PDE4A (cAMP-specific 3′,5′-cyclic phosphodiesterase 4A), PDE4B (Phosphodiesterase 4B cAMP-specific), PDE4B (Phosphodiesterase 4B cAMP-specific), PDE4D (Phosphodiesterase 4D cAMP-specific), PDGFA (Platelet-derived growth factor alpha polypeptide), PDGFB (Platelet-derived growth factor beta polypeptide), PDGFC (Platelet derived growth factor C), PDGFRB (Beta-type platelet-derived growth factor receptor), PDPN (Podoplanin), PENK (Enkephalin), PER1 (Period homolog 1), PLA2 (Phospholipase A2), PLAU (Plasminogen activator urokinase), PLXNC1 (Plexin C1), PMVK (Phosphomevalonate kinase), PNOC (Prepronociceptin), POLH (Polymerase (DNA directed) eta), POMC (Proopiomelanocortin (adrenocorticotropin/beta-lipotropin/alpha-melanocyte stimulating hormone/beta-melanocyte stimulating hormone/beta-endorphin)), POU2AF1 (POU domain class 2 associating factor 1), PRKAA1 (5′-AMP-activated protein kinase catalytic subunit alpha-1), PRL (Prolactin), PSCDBP (Cytohesin 1 interacting protein), PSPN (Persephin), PTAFR (Platelet-activating factor receptor), PTGS2 (Prostaglandin-endoperoxide synthase 2), PTN (Pleiotrophin), PTPN11 (Protein tyrosine phosphatase non-receptor type 11), PYY (Peptide YY), RAB11B (RAB11B member RAS oncogene family), RAB6A (RAB6A member RAS oncogene family), RAD17 (RAD17 homolog), RAF1 (RAF proto-oncogene serine/threonine-protein kinase), RANBP2 (RAN binding protein 2), RAP1A (RAP1A member of RAS oncogene family), RB1 (Retinoblastoma 1), RBL2 (Retinoblastoma-like 2 (p130)), RCVRN (Recoverin), REM2 (RAS/RAD/GEM-like GTP binding 2), RFRP (RFamide-related peptide), RPS6KA3 (Ribosomal protein S6 kinase 90 kDa polypeptide 3), RTN4 (Reticulon 4), RUNX1 (Runt-related transcription factor 1), S100A4 (S100 calcium binding protein A4), S1PR1 (Sphingosine-1-phosphate receptor 1), SCG2 (Secretogranin II), SCYE1 (Small inducible cytokine subfamily E member 1), SELENBP1 (Selenium binding protein 1), SGK (Serum/glucocorticoid regulated kinase), SKD1 (Suppressor of K+ transport growth defect 1), SLC14A1 (Solute carrier family 14 (urea transporter) member 1 (Kidd blood group)), SLC25A37 (Solute carrier family 25 member 37), SMAD2 (SMAD family member 2), SMAD5 (SMAD family member 5), SNAP23 (Synaptosomal-associated protein 23 kDa), SNCB (Synuclein beta), SNF1LK (SNF1-like kinase), SORT1 (Sortilin 1), SSB (Sjogren syndrome antigen B), STAT1 (Signal transducer and activator of transcription 1, 91 kDa), STAT5A (Signal transducer and activator of transcription 5A), STAT5B (Signal transducer and activator of transcription 5B), STX16 (Syntaxin 16), TAC1 (Tachykinin precursor 1), TBX1 (T-box 1), TEF (Thyrotrophic embryonic factor), TF (Transferrin), TGFA (Transforming growth factor alpha), TGFB1 (Transforming growth factor beta 1), TGFB2 (Transforming growth factor beta 2), TGFB3 (Transforming growth factor beta 3), TGFBR1 (Transforming growth factor beta receptor I), TGM2 (Transglutaminase 2), THPO (Thrombopoietin), TIMP1 (TIMP metallopeptidase inhibitor 1), TIMP3 (TIMP metallopeptidase inhibitor 3), TMEM129 (Transmembrane protein 129), TNFRC6 (TNFR/NGFR cysteine-rich region), TNFRSF10A (Tumor necrosis factor receptor superfamily member 10a), TNFRSF10C (Tumor necrosis factor receptor superfamily member 10c decoy without an intracellular domain), TNFRSF1A (Tumor necrosis factor receptor superfamily member 1A), TOB2 (Transducer of ERBB2 2), TOP1 (Topoisomerase (DNA) I), TOPOII (Topoisomerase 2), TRAK2 (Trafficking protein kinesin binding 2), TRH (Thyrotropin-releasing hormone), TSH (Thyroid-stimulating hormone alpha), TUBA1A (Tubulin alpha 1a), TXK (TXK tyrosine kinase), TYK2 (Tyrosine kinase 2), UCP1 (Uncoupling protein 1), UCP2 (Uncoupling protein 2), ULIP (Unc-33-like phosphoprotein), UTRN (Utrophin), VEGF (Vascular endothelial growth factor), VGF (VGF nerve growth factor inducible), VIP (Vasoactive intestinal peptide), VNN1 (Vanin 1), VTN (Vitronectin), WNT2 (Wingless-type MMTV integration site family member 2), XRCC6 (X-ray repair cross-complementing 6), ZEB2 (Zinc finger E-box binding homeobox 2), and ZNF461 (Zinc finger protein 461).

Preferred prion disorder-related proteins, as illustrated above, include PrP^{C} and isoforms thereof, PrP^{Sc} and isoforms thereof, HECTD2 (e3-ubipuitin ligase protein), STI1 (stress inducible protein 1), DPL (residue Doppel protein, encoded by Prnd), APOA1 (Apolipoprotein A1), BCL-2 (B-cell lymphoma 2), HSP60 (Heat shock 60 kDa protein), BAX-inhibiting peptide (Bcl-2-associated X protein inhibitor), NRF2 (nuclear respiratory factor 2), NCAMs (neural cell-adhesion molecules), heparin, laminin and laminin receptor and any combination thereof.

(b) animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

### (c) Prion Disorder-Related Protein

The prion disorder-related protein may be from any of the animals listed above, and may be isoforms or any related protein. Furthermore, the prion disorder-related protein may be a human prion disorder-related protein. Additionally, the prion disorder-related protein may be a bacterial, fungal, or plant prion disorder-related protein. The type of animal and the source of the protein can and will vary. The protein may be endogenous or exogenous (such as an orthologous protein). As an example, the genetically modified animal may be a rat, cat, dog, or pig, and the orthologous prion disorder-related protein may be human. Alternatively, the genetically modified animal may be a rat, cat, or pig, and the orthologous prion disorder-related protein may be canine. One of skill in the art will readily appreciate that numerous combinations are possible.

Additionally, the prion disorder-related protein encoding gene may be modified to include a tag or reporter gene or genes as are well-known. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence encoding a prion disorder-related protein. The genetically modified cell or cell line may be derived from any of the genetically modified animals disclosed herein. Alternatively, the chromosomal sequence coding a prion disorder-related protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:I538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences encoding prion disorder-related proteins may further comprise introducing at least one donor polynucleotide comprising a sequence encoding a prion disorder-related protein into the embryo or cell. A donor polynucleotide comprises at least three components: the sequence coding the prion disorder-related protein, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a prion disorder-related protein may be a BAC.

The sequence of the donor polynucleotide that encodes the prion disorder-related protein may include coding (i.e., exon) sequence, as well as intron sequences and upstream regulatory sequences (such as, e.g., a promoter). Depending upon the identity and the source of the prion disorder-related protein, the size of the sequence encoding the prion disorder-related protein can and will vary. For example, the sequence encoding the prion disorder-related protein may range in size from about 1 kb to about 5,000 kb.

The donor polynucleotide also comprises upstream and downstream sequence flanking the sequence encoding the prion disorder-related protein. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the prion disorder-related protein, a double stranded break introduced into the chromosomal sequence by the zinc finger nuclease is repaired, via homologous recombination with the donor polynucleotide, such that the sequence encoding the prion disorder-related protein is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the sequence encoding the prion disorder-related protein as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences encoding prion disorder-related protein may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the prion disorder-related protein in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated prnd chromosomal sequence may be crossed with animal B comprising a chromosomally integrated sequence encoding a human Dpl protein to give rise to a “humanized” Dpl offspring comprising both the inactivated prnd chromosomal sequence and the chromosomally integrated human prnd sequence. Similarly, an animal comprising an inactivated prnd hectd2 chromosomal sequence may be crossed with an animal comprising a chromosomally integrated sequence encoding the human prion disorder-related HECTD2 protein to generate “humanized” prion disorder-related HECTD2 offspring. Moreover, a humanized Dpl animal may be crossed with a humanized HECTD2 animal to create a humanized Dpl/HECTD2. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include without limit nucleic acids encoding drug transporter proteins, Mdr protein, and the like.

### IV. Applications

A further aspect of the present disclosure encompasses a method for assessing at least one effect of an agent. Suitable agents include without limit pharmaceutically active ingredients, drugs, food additives, pesticides, herbicides, toxins, industrial chemicals, household chemicals, and other environmental chemicals. For example, the effect of an agent may be measured in a “humanized” genetically modified animal, such that the information gained therefrom may be used to predict the effect of the agent in a human. In general, the method comprises contacting a genetically modified animal comprising at least one inactivated chromosomal sequence encoding a prion disorder-related protein and at least one chromosomally integrated sequence encoding an orthologous prion disorder-related protein with the agent, and comparing results of a selected parameter to results obtained from contacting a wild-type animal with the same agent. Selected parameters include but are not limited to (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); and (i) extrahepatic contribution to metabolic rate and clearance of the agent or its metabolite(s).

An additional aspect provides a method for assessing the therapeutic potential of an agent in an animal that may include contacting a genetically modified animal comprising at least one edited chromosomal sequence encoding a prion disorder-related protein, and comparing results of a selected parameter to results obtained from a wild-type animal with no contact with the same agent, Selected parameters include but are not limited to a) spontaneous behaviors; b) performance during behavioral testing; c) physiological anomalies; d) abnormalities in tissues or cells; e) biochemical function; and f) molecular structures.

Also provided are methods to assess the effect(s) of an agent in an isolated cell comprising at least one edited chromosomal sequence encoding a prion disorder-related protein, as well as methods of using lysates of such cells (or cells derived from a genetically modified animal disclosed herein) to assess the effect(s) of an agent. For example, the role of a particular prion disorder-related protein in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy strategy. That is, a chromosomal sequence encoding a prion disorder-related protein may be modified such that the potential of prion infection is reduced or eliminated. In particular, the method comprises editing a chromosomal sequence encoding a prion disorder-related protein such that an altered protein product is produced. Consequently, the therapeutic potential of the prion disorder-related gene therapy regime may be assessed.

Still yet another aspect encompasses a method of generating a cell line or cell lysate using a genetically modified animal comprising an edited chromosomal sequence encoding a prion disorder-related protein. An additional other aspect encompasses a method of producing purified biological components using a genetically modified cell or animal comprising an edited chromosomal sequence encoding a prion disorder-related protein. Non-limiting examples of biological components include antibodies, cytokines, signal proteins, enzymes, receptor agonists and receptor antagonists.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### Examples

The following examples are included to illustrate the invention.

### Example 1

### Genome Editing of the prnd Locus

Zinc finger nucleases (ZFNs) that target and cleave the prdn locus of rats may be designed, assembled, and validated using strategies and procedures previously described (see Geurts et al. Science (2009) 325:433). ZFN design made use of an archive of pre-validated 1-finger and 2-finger modules. The rat prdn gene region may be scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding pairs of ZFNs may be produced using known molecular biology techniques. The mRNA may be transfected into rat cells. Control cells may be injected with mRNA encoding GFP. Active ZFN pairs may be identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch will be cleaved by the surveyor nuclease Cel-1, and the cleavage products can be resolved by gel electrophoresis. This assay may identify a pair of active ZFNs that edited the prnd locus.

To mediate editing of the prnd gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA can be isolated using standard procedures. The targeted region of the prnd locus is to be PCR amplified using appropriate primers. The amplified DNA is to be subcloned into a suitable vector and sequenced using standard methods.

### Example 2

### Genome Editing of Dpl Genes in Model Organism Cells

ZFN-mediated genome editing may be used to study the effects of a “knockout” mutation in an AD-related chromosomal sequence, such as a chromosomal sequence encoding the Dpl protein, in a genetically modified model animal and cells derived from the animal. Such a model animal may be a rat. In general, ZFNs that bind to the rat chromosomal sequence encoding the Dpl protein associated with AD may be used to introduce a deletion or insertion such that the coding region of the Dpl gene (Prnd) is disrupted such that a functional Dpl protein may not be produced.

Suitable fertilized embryos may be microinjected with capped, polyadenylated mRNA encoding the ZFN essentially as detailed above in Example 1. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the Dpl “knockout” may be assessed in the genetically modified rat or progeny thereof. Furthermore, molecular analyses of AD-related pathways may be performed in cells derived from the genetically modified animal comprising a Dpl “knockout”.

### Example 3

### Genome Editing of Prp Genes in Model Organisms

Coding polymorphism at PrP codon 129 has a strong association with disease susceptibility and phenotype modifying effect, especially when the amino acid at codon 129 is methionine or valine. ZFN-mediated genome editing may be used to generate a humanized rat wherein the rat Prp gene is replaced with a mutant form of the human Prpn gene comprising sequence with 129M or 129V. Such a humanized rat may be used to study the development of the diseases associated with the mutant human PSEN2 protein. In addition, the humanized rat may be used to assess the efficacy of potential therapeutic agents targeted at the pathway leading to prion disorder comprising neurotoxic PrP isoform.

What is claimed is:

1. A genetically modified animal comprising at least one edited chromosomal sequence encoding a prion disorder-related protein.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no functional prion disorder-related protein is produced.

4. The genetically modified animal of claim 3, wherein inactivated chromosomal sequence comprises no exogenously introduced sequence.

5. The genetically modified animal of claim 3, further comprising at least one chromosomally integrated sequence encoding a functional prion disorder-related protein.

6. The genetically modified animal of claim 1, wherein the prion disorder-related protein is chosen from PrP, HECTD2, STL, APOA1, BCL-2, HSP60, BAX-inhibiting peptide, NRF2, NCAMs, Heparin, laminin, laminin receptor, and combinations thereof.

7. The genetically modified animal of claim 1, further comprising a conditional knock-out system for conditional expression of the prion disorder-related protein.

8. The genetically modified animal of claim 1, wherein the edited chromosomal sequence comprises an integrated reporter sequence.

9. The genetically modified animal of claim 1, wherein the animal is heterozygous or homozygous for the at least one edited chromosomal sequence.

10. The genetically modified animal of claim 1, wherein the animal is an embryo, a juvenile, or an adult.

11. The genetically modified animal of claim 1, wherein the animal is chosen from bovine, canine, equine, feline, ovine, porcine, non-human primate, and rodent.

12. The genetically modified animal of claim 1, wherein the animal is rat.

13. The genetically modified animal of claim 1, wherein the animal is rat and the protein is a human prion disorder-related protein.

14. A non-human embryo, the embryo comprising at least one RNA molecule encoding a zinc finger nuclease that recognizes a chromosomal sequence encoding a prion disorder-related protein, and, optionally, at least one donor polynucleotide comprising a sequence encoding a prion disorder-related protein.

15. The non-human embryo of claim 14, wherein the prion disorder-related protein is chosen from PrP, HECTD2, STL, APOA1, BCL-2, HSP60, BAX-inhibiting peptide, NRF2, NCAMs, Heparin, laminin, laminin receptor, and combinations thereof.

16. The non-human embryo of claim 14, wherein the embryo is chosen from bovine, canine, equine, feline, ovine, porcine, non-human primate, and rodent.

17. The non-human embryo of claim 14, wherein the embryo is rat and the protein is an ortholog of a human prion disorder-related protein.

18. A genetically modified cell, the cell comprising at least one edited chromosomal sequence encoding a prion disorder-related protein.

19. The genetically modified cell of claim 18, wherein the edited chromosomal sequence is inactivated, modified, or comprises an integrated sequence.

20. The genetically modified cell of claim 19, wherein the edited chromosomal sequence is inactivated such that a functional prion disorder-related protein is not produced.

21. The genetically modified cell of claim 20, further comprising at least one chromosomally integrated sequence encoding a functional prion disorder-related protein.

22. The genetically modified cell of claim 18, wherein the prion disorder-related protein is chosen from PrP, HECTD2, STL, APOA1, BCL-2, HSP60, BAX-inhibiting peptide, NRF2, NCAMs, Heparin, Laminin, Laminin receptor, and combinations thereof.

23. The genetically modified cell of claim 18, wherein the cell is of bovine, canine, equine, feline, human, ovine, porcine, non-human primate, or rodent origin.

24. The genetically modified cell of claim 18, wherein the cell is heterozygous or homozygous for the at least one edited chromosomal sequence.

25. The genetically modified cell of claim 18, wherein the cell is of rat origin and the protein is a human prion disorder-related protein.

26. The genetically modified cell of claim 20, wherein inactivated chromosomal sequence comprises no exogenously introduced sequence.

27. The genetically modified cell of claim 18, further comprising a conditional knock-out system for conditional expression of the prion disorder-related protein.

28. The genetically modified cell of claim 18, wherein the edited chromosomal sequence comprises an integrated reporter sequence.

29. A method for assessing the effect of an agent in an animal, the method comprising contacting a genetically modified animal comprising at least one edited chromosomal sequence encoding a prion disorder-related protein with the agent, and comparing results of a selected parameter to results obtained from contacting a wild-type animal with the same agent, wherein the selected parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) efficacy of the agent or its metabolite(s).

30. The method of claim 29, wherein the agent is a pharmaceutically active ingredient, a drug, a toxin, or a chemical.

31. The method of claim 29, wherein the at least one edited chromosomal sequence is inactivated such that the prion disorder-related protein is not produced or is not functional, and wherein the animal further comprises at least one chromosomally integrated sequence encoding a functional ortholog of the prion disorder-related protein.

32. The method of claim 29, wherein the prion disorder-related protein is chosen from PrP, HECTD2, STL, APOA1, BCL-2, HSP60, BAX-inhibiting peptide, NRF2, NCAMs, Heparin, laminin, laminin receptor, and combinations thereof.

33. A method for assessing the therapeutic potential of an agent in an animal, the method comprising contacting a genetically modified animal comprising at least one edited chromosomal sequence encoding a prion disorder-related protein with an agent, and comparing results of a selected parameter to results obtained from a wild-type animal with no contact with the same agent, wherein the selected parameter is chosen from:
a) spontaneous behaviors;
b) performance during behavioral testing;
c) physiological anomalies;
d) abnormalities in tissues or cells;
e) biochemical function; and
f) molecular structures.

34. The method of claim 33, wherein the agent is a pharmaceutically active ingredient, a drug, a toxin, a biologically active agent, or a chemical.

35. The method of claim 33, wherein the at least one edited chromosomal sequence is inactivated such that the prion disorder-related protein is not produced or is not functional, and wherein the animal further comprises at least one chromosomally integrated sequence encoding a functional ortholog of the prion disorder-related protein.

36. The method of claim 33, wherein the prion disorder-related protein is chosen from PrP, HECTD2, STL, APOA1, BCL-2, HSP60, BAX-inhibiting peptide, NRF2, NCAMs, Heparin, laminin, laminin receptor, and combinations thereof.
